(19) United States
(12) Patent Application Publication
(10) Pub. No.: US 2018/0049711 A1
(43) Pub. Date: Feb. 22, 2018

(54) METHOD OF PANORAMIC IMAGING WITH A DUAL PLANE FLUOROSCOPY SYSTEM

(71) Applicant: Whale Imaging, Inc., Waltham, MA (US)

(72) Inventors: Changguo Ji, Lexington, MA (US); Xingbai He, Belmont, MA (US); Chun He, Beijing (CN); William Wong, Milton, MA (US)

(21) Appl. No.: 15/680,867

(22) Filed: Aug. 18, 2017

Related U.S. Application Data
(60) Provisional application No. 62/377,469, filed on Aug. 19, 2016.

(51) Int. Cl. A61B 6/00; G06T 7/246; G06T 3/40

(52) U.S. Cl. CPC A61B 6/487; G06T 7/248; G06T 3/4038; H04N 5/23238; A61B 6/547; A61B 6/4435; A61B 6/4405; A61B 6/5241

(57) ABSTRACT

A method and system for creating non-parallax panoramic images from a plurality of individual images, in real-time is provided. Specifically, the present invention provides a system and method configured to combine individual overlapping medical images into a single undistorted panoramic image in real-time as an imaging device traverses over a patient. In particular, the present invention provides a system and method for combining individual x-ray images into a single clinical panoramic image for use with a traversing G-arm device.

### CROSS-REFERENCE TO RELATED APPLICATION(S)

This application claims priority to, and the benefit of, co-pending U.S. Provisional Application No. 62/377,469, filed Aug. 19, 2016, for all subject matter common to both applications. The disclosure of said provisional application is hereby incorporated by reference in its entirety.

### FIELD OF THE INVENTION

The present invention relates to an imaging system and method for producing a panoramic image for use during a fluoroscopic procedure. In particular, the present invention relates to fluoroscopic imaging system configured to create a single non-parallax panoramic image in real-time from a plurality of individual images captured by the imaging system while the imaging system is traversing over a patient.

### BACKGROUND

Generally, the usage of conventional C-arm X-ray equipment is well known in the medical art of surgical and other interventional procedures. Traditionally, the utilization of C-arm X-ray equipment enables flexibility in operation procedures and in the positioning process, which is reflected by a number of degrees of freedom of movement provided by the C-arm X-ray equipment.

In a conventional implementation, a C-arm gantry is slidably mounted to a support structure to enable orbiting rotational movement of the C-arm about a center of curvature for the C-arm. Additionally, the C-arm equipment provides a lateral rotation motion rotating along the horizontal axis of the support structure. Moreover, the C-arm equipment also can include an up-down motion along the vertical axis, a cross-arm motion along the horizontal axis, and a wig-wag motion along the vertical axis.

A traditional C-arm provides real time X-ray images of a patient's spinal anatomy which is used to guide a surgeon during an operating procedure. For example, spinal deformity correction is a type of surgery that frequently uses the C-arm during an operation procedure. Such surgeries typically involve corrective manoeuvres to improve the sagittal or coronal profile of the patient. However, an intra-operative estimation of the amount of correction is difficult. Mostly, anteroposterior (AP) and lateral fluoroscopic images are used, but are limited as the AP and lateral fluoroscopic images only depict a small portion of the spine in a single C-arm image. The small depiction of the spine in traditional C-arm images is due to the limited field of view of a C-arm machine. As a result, spine surgeons are missing an effective tool to image an entire spine of a patient for use during surgery and assessing the extent of correction in scoliotic deformity.

Similarly, the full bone structure of X-ray images cannot be captured in a single scan with existing Digital radiography (DR) systems. Stitching methods and systems for X-ray images is very important for scoliosis or lower limb malformation diagnosing and pre-surgical planning. Although radiographs that are obtained either by using a large field detector or by image stitching can be used to image an entire spine, the radiographs are usually not available for intra-operative interventions because there are not motorized positioning mechanisms available for conventional digital radiography systems along a horizontal positioning of a patient.

One alternative to conventional radiographs is to develop methods and systems to stitch multiple intra-operatively acquired small fluoroscopic images together to be able to display the entire spine at once. It has been known that there are a few methods to create a single panoramic image of a long view using C-arm from several individual fluoroscopic X-ray images. Panoramic images are useful preoperatively for diagnosis, and intra-operatively for long bone fragment alignment, for making anatomical measurements, and for documenting surgical outcomes. (See, U.S. Patent Application No. 2011/0188726) U.S. Patent Application No. 2011/0188726 disclosed a method for generating a panoramic image of a region of interest (ROI) which is larger than a field of a view of a radiation based imaging device, including, positioning markers along the ROI, acquiring a set of images along the ROI, wherein the acquired images have at least partially overlapping portions, aligning at least two separate images by aligning a common marker found in both images and compensating for a difference between a distance from a radiation source to the marker element and the distance from the radiation source to a plane of interest.

Although the C-arm X-ray equipment is smart and flexible in positioning process, it is often desirable to take X-rays of a patient from both the AP and LAT positions (two perpendicular angles). In such situations, the operators have to reposition the C-arm because C-arm configurations do not allow for such perpendicular bi-planar imaging. For taking the X-rays from different angles at the same time without repositioning the X-ray apparatus, such a configuration is often referred to as bi-planar imaging, also known as G-arm or G-shape arm (see U.S. Pat. No. 8,992,082), that allows an object to be viewed in two planes simultaneously. The two X-ray beams emitted from the two X-ray tubes may cross at an iso-center.

A traditional mobile dual plane fluoroscopy device has advantages of each of C-shaped, G-shaped, and ring-shaped arm configurations. The device consists of a gantry that supports X-ray imaging machinery. The gantry is formed to allow two bi-planar X-rays to be taken simultaneously or without movement of the equipment and/or patient. The gantry is adjustable to change angles of the X-ray imaging machinery. In addition, the X-ray receptor portion of the X-ray imaging machinery may be positioned on retractable and extendable arms, allowing the apparatus to have a larger access opening when not in operation, but to still provide bi-planar X-ray ability when in operation.

### SUMMARY

There is a need for improvements to producing a panoramic image of a patient subject, in real-time, during a medical procedure. The present invention is directed toward further solutions to address this need, in addition to having other desirable characteristics. Specifically, the present invention provides a system and method that is configured to capture a plurality of image frames while an imaging device traverses over a patient, transforms the plurality of image frames into a single non-parallax panoramic image, and generates and displays the panoramic image to an operating user in real-time. The present invention is adapted to find the overlapping region of a plurality of images, utilize a correlation coefficient to evaluate similarities of overlapping region(s) of the plurality of images, and perform weighted blending to produce the panorama image in real-time.

In accordance with example embodiments of the present invention, a method for panoramic imaging is provided. The method includes activating an imaging system. The system includes a first imaging assembly mounted on a support gantry, the first imaging assembly configured to capture image data comprising a plurality of image frames. The system also includes a control unit that directs movement and positioning of the support gantry and a processing and display device in communication with the first imaging assembly, the processing and display device configured to stitch and display the plurality of image frames as a single panoramic image. The method also includes traversing the support gantry, via the control unit, parallel to a subject to be imaged. The processing and display device constructs and displays the panoramic image in real-time based on the traversing of the support gantry and the plurality of image frames obtained during the traversing.

In accordance with aspects of the present invention, the stitching includes calculating a motion between image frames along an X-axis of the traversing the support gantry and determining a size of an original panoramic image based on the calculated motion between image frames. The stitching also includes downsampling the original panoramic image along the X-axis, determining a downsample size of the original panoramic image based on the downsampling, and downsampling the original panoramic image along the Y-axis. The downsampling can also include selecting two or more overlapping lines between adjacent image frames, determining the traversing speed of the support gantry, performing a weighted operation to reduce parallax error, interpolating each of the two or more overlapping lines, and normalizing and summing the interpolated overlapping lines. The weighted operation can be a Gaussian formula of: Wᵢ₍ₖ₎=e^{-0.5(Dist(k)/64)}^{²}, where Wi(k) is a Gaussian weight and Dist(k) is a distance between overlapping lines for image frames. The downsampling can be performed without a weighted operation.

In accordance with aspects of the present invention, the stitching comprises identifying a correlation of overlapping region(s) from adjacent images to find an inter frame motion. The identification of the correlation of overlapping region(s) can include selecting a search area size for searching for a correlation within each of the plurality of image frames, such that the search area size is smaller than a single image frame size, selecting a pattern image size to be used in the correlation search, such that the pattern image size is smaller than the search area size, comparing a pattern image to an area defined by the search area size for each of the plurality of image frames, and determining a correlation value based on the comparing.

In accordance with aspects of the present invention, the first imaging assembly comprises a first imaging energy emitter that is positioned opposite a first imaging receptor, such that one of the first imaging energy emitter or the first imaging receptor is positioned at the first terminal end of the support gantry. The imaging system can further include a second imaging assembly that is positioned on the support gantry and comprising a second imaging energy emitter positioned that is opposite a second imaging receptor, such that one of the second imaging energy emitter or the second imaging receptor is positioned at the second terminal end of the support gantry.

In accordance with aspects of the present invention, the tracking wheels configured to enable traversing of the support gantry and tracking a distance traversed by the support gantry.

In accordance with example embodiments of the present invention, a system for panoramic imaging is provided. The system includes a support gantry including a plurality of tracking wheels configured to enable the support gantry to traverse in a single axis direction and a first imaging assembly mounted on the support gantry, the first imaging assembly configured to capture image data comprising a plurality of image frames while the support gantry traverses parallel to a subject. They system also includes a control unit that directs movement and positioning of the support gantry and a processing and display device in communication with the first imaging assembly, the processing and display device configured to stitch and display the plurality of image frames as a single panoramic image. The processing and display device is configured to construct and display the panoramic image in real-time based on a traversing of the support gantry and the plurality of image frames obtained during the traversing.

In accordance with aspects of the present invention, the stitching includes the processing and display device calculating a motion between image frames along an X-axis of the traversing the support gantry, the processing and display device determining a size of an original panoramic image based on the calculated motion between image frames, the processing and display device downsampling the original panoramic image along the X-axis, the processing and display device determining a downsample size of the original panoramic image based on the downsampling, and the processing and display device downsampling the original panoramic image along the Y-axis. The downsampling can include a stitching tool selecting two or more overlapping lines between adjacent image frames, the stitching tool determining the traversing speed of the support gantry, the stitching tool performing a weighted operation to reduce parallax error, the stitching tool interpolating each of the two or more overlapping lines, and the stitching tool normalizing and summing the interpolated overlapping lines. The weighted operation can be a Gaussian formula of: Wᵢ₍ₖ₎=e^{-0.5(Dist(k)/64)}^{²}, where Wi(k) is aGaussian weight and Dist(k) is a distance between overlapping lines for image frames. The stitching tool can perform the downsampling without a weighted operation.

In accordance with aspects of the present invention, the stitching comprises a correlation tool identifying a correlation of overlapping region(s) from adjacent images to find an inter frame motion. The identification of the correlation of overlapping region(s) can include the correlation tool selecting a search area size for searching for a correlation within each of the plurality of image frames, such that the search area size is smaller than a single image frame size, the correlation tool selecting a pattern image size to be used in the correlation search, such that the pattern image size is smaller than the search area size, the correlation tool comparing a pattern image to an area defined by the search area size for each of the plurality of image frames, and the correlation tool determining a correlation value based on the comparing.

In accordance with aspects of the present invention, the first imaging assembly includes a first imaging energy emitter that is positioned opposite a first imaging receptor, such that one of the first imaging energy emitter or the first imaging receptor is positioned at the first terminal end of the support gantry. The imaging system can further include a second imaging assembly that is positioned on the support gantry and comprising a second imaging energy emitter positioned that is opposite a second imaging receptor, such that one of the second imaging energy emitter or the second imaging receptor is positioned at the second terminal end of the support gantry.

In accordance with aspects of the present invention, the tracking wheels are configured to track a distance traversed by the support gantry.

### BRIEF DESCRIPTION OF THE FIGURES

These and other characteristics of the present invention will be more fully understood by reference to the following detailed description in conjunction with the attached drawings, in which:

FIG. 1 is a diagrammatic illustration depicting the main components of a conventional G-arm medical imaging system in accordance with the present invention;

FIG. 2 is a diagrammatic illustration of a system for implementation of the present invention;

FIG. 3 is an example illustration of a plurality of image frames and a panoramic image created from the plurality of image frames, in accordance with the present invention;

FIG. 4 is a flowchart depicting an example stitching operation performed by the imaging system, in accordance with aspects of the present invention;

FIG. 5 is a diagrammatic illustration of identifying correlations between overlapping image frames, in accordance with aspects of the present invention;

FIG. 6 is a flowchart depicting an example correlation operation provided by the imaging system, in accordance with aspects of the present invention;

FIG. 7 is a flowchart depicting an example operation of the imaging system, in accordance with aspects of the present invention; and

FIG. 8 is a diagrammatic illustration of a high level architecture for implementing processes in accordance with aspects of the present invention.

### DETAILED DESCRIPTION

An illustrative embodiment of the present invention relates to a method and system for combining individual overlapping medical images into a single undistorted panoramic image in real-time. The present invention identifies overlapping fields of view between a plurality of images, such that the overlaps can be used in a digital stitching process to create a digital panoramic image. Specifically, the present invention provides an image correlation algorithm for fine inter-frame translation that is adapted to find the overlapping region of a plurality of images, a correlation coefficient that is used to evaluate the similarity of overlapping region(s) of the plurality of images, and weighted blending is performed to produce a panorama image. In accordance with an example embodiment of the present invention, the weighted blending is the contribution factor of a pixel in a sub-image to panoramic/stitching image.

The combination of elements utilized in the present invention provide an optimized stitching implementation that is fast enough for real-time stitching and displaying of a digital panoramic image of a patient while the imaging system is moving along the patient. Additionally, the present invention produces robust and accurate panoramic images with quality and spatial resolution that is comparable to that of the individual images, without the utilization of down-sampling and masking to decrease the size of image and reduce the amount of computation (as required in traditional stitching methods and systems). The present invention, however, can utilize down-sampling and masking to further optimize and increase the speed of the stitching process, if desired but it is not required for the present invention to operate effectively. The combination of benefits and functionality provided by the present invention make the invention ideal for use in real-time during a fluoroscopic procedure. The real-time panoramic images provided by the present invention improve the effectiveness, reliability, and accuracy of the user performing the fluoroscopic procedure.

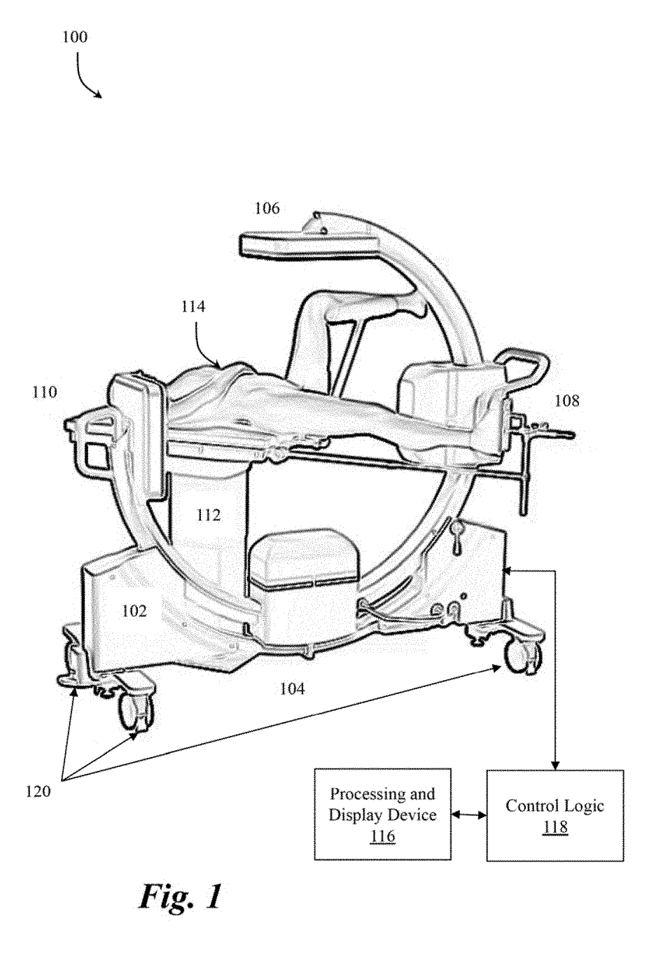

FIGS. 1 through 8, wherein like parts are designated by like reference numerals throughout, illustrate an example embodiment or embodiments of an improved system for creating real-time panoramic images during a fluoroscopic procedure, according to the present invention. Although the present invention will be described with reference to the example embodiment or embodiments illustrated in the figures, it should be understood that many alternative forms can embody the present invention. One of skill in the art will additionally appreciate different ways to alter the parameters of the embodiment(s) disclosed, such as the size, shape, or type of elements or materials, in a manner still in keeping with the spirit and scope of the present invention.

The present invention includes a system and method for implementation with a medical imaging device. In particular, the present invention is configured to produce real-time panoramic images for use during medical procedures (e.g., such as fluoroscopic imaging procedures). As would be appreciated by one skilled in the art, imaging during a procedure can be implemented utilizing a collection of different imaging systems (e.g., C-arm, G-arm bi-plane fluoroscopic imager, etc.). An example of an imaging system for use in accordance with the present invention is depicted in FIG. 1. In particular, FIG. 1 depicts the main components of a G-arm medical imaging system 100 which can be utilized during a fluoroscopic procedure. Although the imaging system 100 depicted in FIG. 1 is a G-arm medical imaging system 100, the illustrative example of a G-arm is for example purposes only and is not intended to limit the present invention to implementation with a G-arm device. For example, the present invention can be implemented on a C-arm or other imaging device.

Continuing with FIG. 1, the main components of the imaging system 100 include a movable stand or support gantry 102 (e.g., via tracking wheels 120), a radiation source 104 and radiation detector 106 configured for a frontal view (or anteroposterior view), a radiation source 108 (e.g., X-ray source) and radiation detector 110 (fluoroscopic imager or X-ray photon detector) configured for a lateral view, and a patient table 112 configured to hold a patient between the radiation sources 104, 108 and the radiation detectors 106, 110. The movable stand or support gantry 102 provides the foundational framework in which each of the other components depicted in FIG. 1 are attached to create the imaging system 100. The radiation sources 104, 108 are configured to produce radiation (e.g., X-ray photons) for projection through a subject patient 114 (e.g., a patient) positioned on a patient table 112. As would be appreciated by one skilled in the art, the radiation sources 104, 108 can include any kind of radiation sources utilized for imaging a patient. For example, the radiation sources 104, 108 can be electromagnetic radiation or x-radiation sources configured to produce X-rays.

In accordance with an example embodiment of the present invention, the imaging system 100 includes or is otherwise communicatively attached to a processing and display device 116 and a control logic device 118. The control logic 118 is configured to receive input from the processing and display device 116 (e.g., via an input from a user) and transmit signals to control the radiation sources 104, 108. In particular, the control logic 118 provides signals for operating the radiation sources 104, 108 and when to produce radiation. The radiation detectors 106, 110 are configured to electrically transform the received radiation, produced by the radiation sources 104, 108, into detectable signals (e.g., raw image data). An example of a traditional radiation detector is a flat panel detector, which is a thin film transistor (TFT) panel with a scintillation material layer configured to receive energy from visible photons to charge capacitors of pixel cells within the TFT panel. The charges for each of the pixel cells are readout as a voltage data value to the processing and display device 206 as an image 216 of the patient (e.g., an X-ray image). As would be appreciated by one skilled in the art, each of the components within the imaging system 100 can include a combination of devices known in the art configured to perform the imaging tasks discussed herein. For example, an image intensifier is an alternative radiation detector that can be utilized in place of the radiation detectors. Additionally, the radiation sources 104, 108 and radiation detectors 106, 110 are positioned in a configuration to simultaneously the capture a posterior image of a patient and a lateral position of the patient (e.g., perpendicular sources and detectors as shown in FIG. 1).

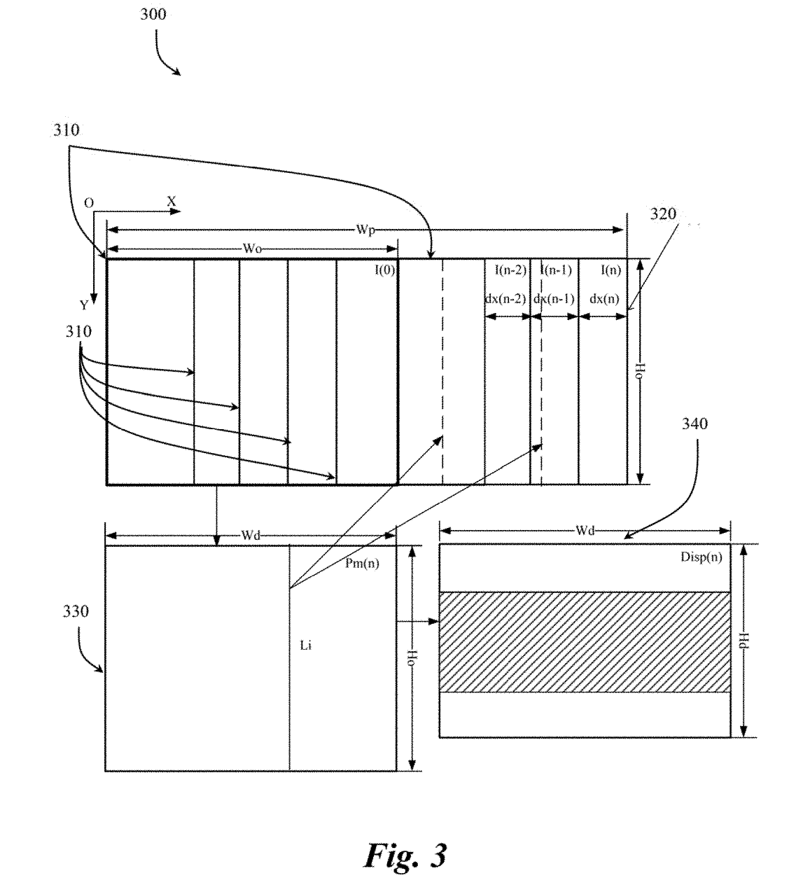

In accordance with an example embodiment of the present invention, the processing and display device 116 is configured to receive the raw image data from the radiation detectors 106, 110, the raw image data including a plurality of limited field of view image frames 310 captured at the different locations at different points in time on a subject patient 312 located between the radiation sources 104, 108 and the radiation detectors 106, 110. In particular, the processing and display device 116 receives the plurality of raw image data captured by the radiation detectors 106, 110 resulting from the radiation sources 104, 108. The processing and display device 116 is configured to transform the raw image data for each of the plurality of image frames 310 into a single panoramic image 340, as discussed in greater detail with respect to FIGS. 3-6. In short, the processing and display device 116 analyzes the plurality of image frames 310 to identify overlaps/correlations between adjacent image frames 310 and stitches together the image frames 310, based on the identified overlaps/correlations into a non-parallax panoramic image. Thereafter, the single panoramic image 340 can be displayed to a user on a display device in real-time.

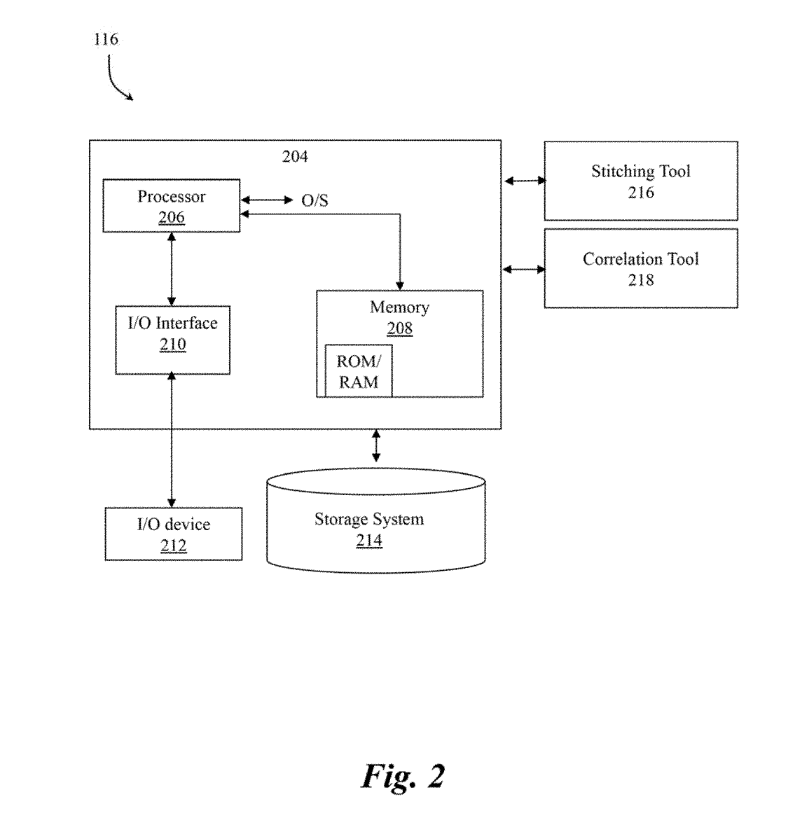

FIG. 2 depicts an illustrative computing system and/or device for implementing the steps in accordance with the aspects of the present invention. In particular, FIG. 2 depicts a computing system and/or device implemented in accordance with the processing and display device 116 for the imaging system 100. In accordance with an example embodiment, the processing and display device 116 is a combination of hardware and software configured to carry out aspects of the present invention. In particular, the processing and display device 116 can include a computing system with specialized software and databases designed for providing a method for optimizing reward-based campaigns. For example, the processing and display device 116 can be software installed on a computing device 204, a web based application provided by a computing device 204 which is accessible by the imaging system 100, a cloud based application accessible by computing devices, or the like. The combination of hardware and software that make up the processing and display device 116 are specifically configured to provide a technical solution to a particular problem utilizing an unconventional combination of steps/operations to carry out aspects of the present invention. In particular, the processing and display device 116 is designed to execute a unique combination of steps to provide a novel approach to stitching together a plurality of medical image frames 310 to produce a single panoramic image in real-time.

In accordance with an example embodiment of the present invention, the processing and display device 116 can include a computing device 204 having a processor 206, a memory 208, an input output interface 210, input and output devices 212 and a storage system 214. Additionally, the computing device 204 can include an operating system configured to carry out operations for the applications installed thereon. As would be appreciated by one skilled in the art, the computing device 204 can include a single computing device, a collection of computing devices in a network computing system, a cloud computing infrastructure, or a combination thereof. Similarly, as would be appreciated by one of skill in the art, the storage system 214 can include any combination of computing devices configured to store and organize a collection of data. For example, storage system 214 can be a local storage device on the computing device 204, a remote database facility, or a cloud computing storage environment. The storage system 214 can also include a database management system utilizing a given database model configured to interact with a user for analyzing the database data.

Continuing with FIG. 2, the processing and display device 116 can include a combination of core components to carry out the various functions of the present invention. In accordance with an example embodiment of the present invention, the processing and display device 116 can include a stitching tool 216 and a correlation tool 218. As would be appreciated by one skilled in the art, the stitching tool 216 and the correlation tool 218 can include any combination of hardware and software configured to carry out the various aspects of the present invention. In particular, each of the stitching tool 216 and the correlation tool 218 are configured to identify correlations between adjacent overlapping image frames 310 and stitching the image frames 310 together into a single panoramic image 340 in real-time.

In accordance with an example embodiment of the present invention, the stitching tool 216 is configured to manage the stitching process in accordance with the present invention. In particular, the stitching tool 216 is configured to receive a plurality of overlapping image frames 310 from the imaging system 100 and create a single panoramic image 340 from the plurality of image frames 310. Any combination of stitching methodologies can be implemented by the stitching tool 216 to create the panoramic image 340. An exemplary example of the stitching process implemented by the stitching tool 216 is discussed in greater detail with respect to FIGS. 3-7.

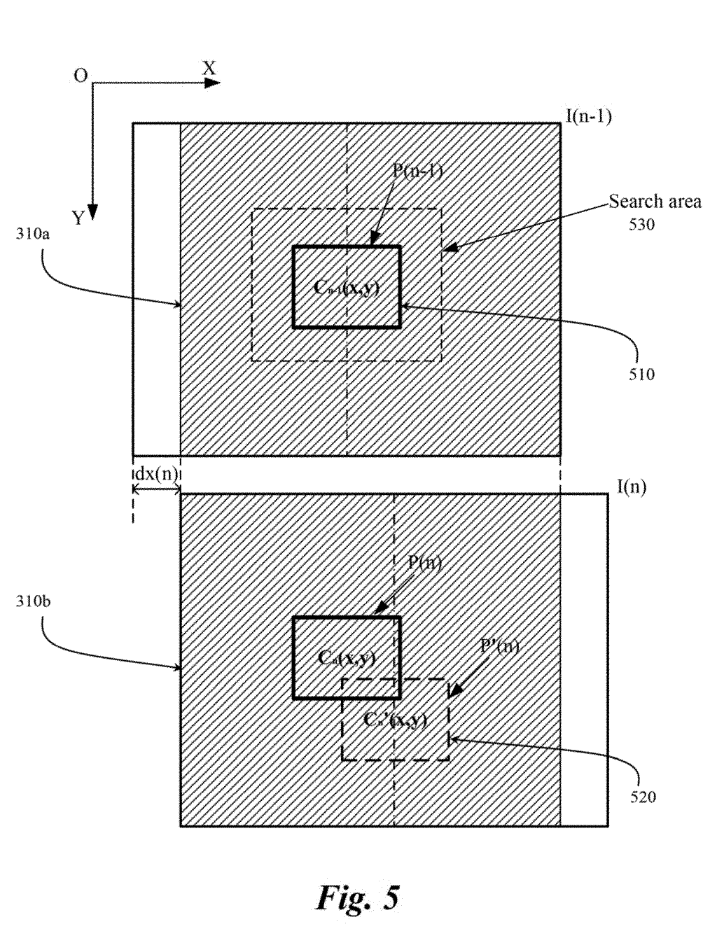

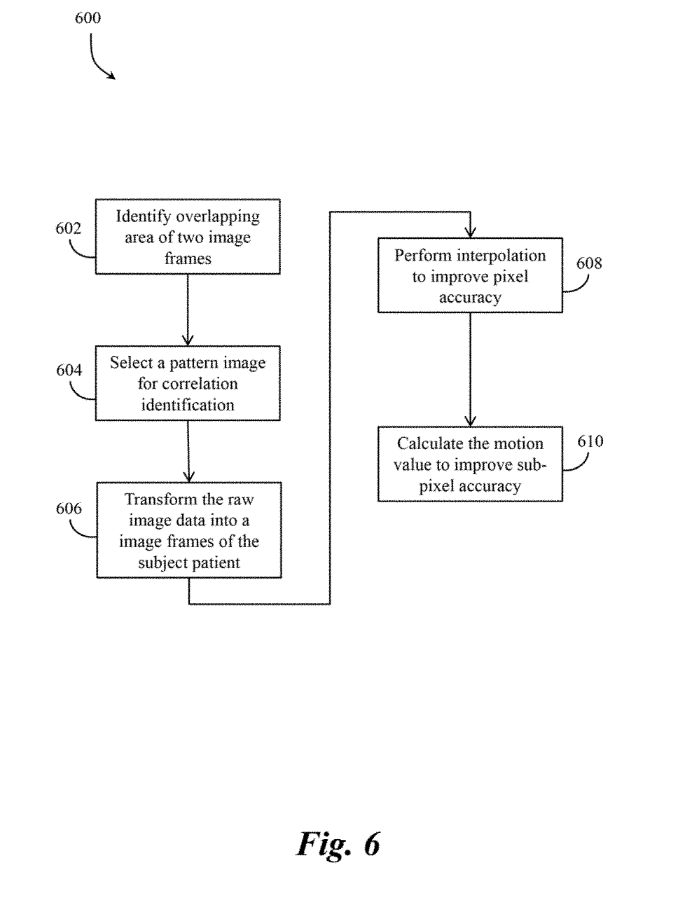

In accordance with an example embodiment of the present invention, the correlation tool 218 is configured to perform a correlation analysis via a correlation algorithm between two adjacent image frames 310. In particular, the correlation tool 218 is configured to find an overlapping region of a plurality of image frames 310 and utilize a correlation coefficient to evaluate the similarities of the overlapping region(s) of the plurality of images. Thereafter, the correlation tool 218 can implement weighted blending to be utilized in the creation of a single panorama image 340 from the plurality of image frames 310. In accordance with an example embodiment of the present invention, the weighted blending is a contribution factor of a pixel in a sub-image to panoramic/stitching image. For example, the correlation tool 218 can utilize a weighting profile, such as triangular, Gaussian, etc., to perform the weighted blending. As would be appreciated by one skilled in the art, any combination of correlation and blending algorithms can be utilized without departing from the scope of the present invention. For example, a correlation can be determined by calculating an overall image intensity difference between two images or using an attenuation map of bone structure to identify a natural marker to find the image translation between individual image frames 310. An illustrative example of the correlation process implemented by the correlation tool 218 is discussed in greater detail with respect to FIGS. 5 and 6.

Returning to FIG. 1, in accordance with an example embodiment of the present invention, the support gantry 102 is configured with tracking wheels 120 to enable the support gantry 102, and the components attached thereto, to be mobile. In particular, the tracking wheels 120 are configured to enable a user to push/pull the support gantry 102 along a single axis for purposes of traversing a span of a subject 114 located within the imaging system 100. Additionally, in accordance with an example embodiment, the tracking wheels 120 and/or a portion of the support gantry 102 can be configured to maintain a traversing path in a single axis. For example, the tracking wheels 120 or another part of the support gantry 102 can be removably attached to a track to ensure that the support gantry 102 only traverses within a single axis (e.g., the X-axis). As would be appreciated by one skilled in the art, any suitable combination of mechanical devices can be utilized to enable the support gantry 102 to be mobile and is not limited to the use of tracking wheels 120. In accordance with an example embodiment of the present invention, the tracking wheels 120 are configured with a tracking mechanism to provide tracking information related to location, distance traveled, speed, etc. The tracking mechanism is further configured to provide the tracking information to the processing and display device 116 for additional processing.

In operation, the imaging system 100 is configured to traverse a length of a subject 114 resting on a patient table 112 of the imaging system 100 (e.g., between the radiation sources 104, 108 and the radiation detectors 106, 110). Additionally, while the imaging system 100 is traversing the length of the subject 114, the imaging system 100 is configured to capture a plurality of independent limited field of view and overlapping image frames 310 representing different portions of the subject 114. In a simplified example, a first image frame 310 can capture a head/shoulder region of a subject 114, a second image frame 310 can capture a torso region of a subject 114, a third image frame 310 can capture a leg region of a subject 114, and so on. Thereafter, the imaging system 100 is configured to transform the overlapping image frames 310 into a single undistorted non-parallax single panoramic image 340 (e.g., a head to toe image of a subject 114) by stitching together the image frames 310.

The imaging system 100 begins the creation of the panoramic imaging process by initiating the radiation sources 104, 108 to generate radiation, directed at and through a patient 114, to be received by radiation detectors 106, 110. In accordance with an example embodiment of the present invention, during generation of the radiation, the support gantry 102 (and the radiation detector 304 attached thereto) traverses in a single axis direction (e.g., in response to pushing/pulling force applied by an operator). For example, the support gantry 102 and the components attached thereto can be pushed/pulled by an operator user (or through an automated mechanical means) causing the support gantry 102 to traverse along fixed path via the tracking wheels 120 on the support gantry 102.

As the support gantry 102 traverses, the radiation detectors 106, 110 attached thereto will receive the radiation generated by the radiation sources 104 and generate periodic readouts of the received radiation (e.g., as raw image data). As would be appreciated by one skilled in the art, as the support gantry 102 is traversing, the corresponding raw image data captured by the radiation detectors 106, 110 will reflect the location of the subject 114 at the traversed location at that point in time. Simultaneous with the support gantry 102 traversing and the radiation detectors 106, 110 providing raw data readouts, the processing and display device 116 receives the raw image data from the radiation detectors 106, 110. Based on the received raw image data, the processing and display device 116 is configured to transform the raw image data into a plurality of viewable image frames 310. As would be appreciated by one skilled in the art, the raw data can be periodically sampled to create data for the plurality of independent image frames 310. In accordance with an example embodiment of the present invention, each transmission of each independent collection of raw data (e.g., for each individual image) includes a respective location of the radiation detector 304 (e.g., according to a mechanical positioning of the support gantry 102/tracking wheels 120) at the time that the raw data was captured.

In accordance with an example embodiment of the present invention, the raw image data is sampled at a rate such that the plurality of image frames 310 are overlapping images. Utilizing the determined motion of the traversing support gantry 102, the processing and display device 306 creates a single non-parallax wide-view panoramic image 340 by stitching together the overlapping plurality of image frames 310, as discussed in greater detail with respect to FIGS. 3-6. In accordance with an example embodiment of the present invention, the non-parallax panoramic image is stitched together based on identifying correlations between adjacent image frames 310. In particular, the panoramic image is created by identifying the overlapping regions of the plurality of images and interpolating the images from an adjacent view utilizing a weighting profile. For example, the processing and display device 116 can utilize a Gaussian or triangular weighting profile to create the panoramic image. The imaging system 100 can update the panoramic image 340 on the fly in real-time as image frames 310 are received and stitched together with the previously received image frames 310.

FIG. 3 depicts an exemplary representation of a plurality of overlapping image frames 310 captured by the imaging system 100 and to be utilized to create a single non-parallax panoramic image (e.g., via a stitching process). In particular, FIG. 3 depicts a plurality of overlapping image frames 310 starting with the initial image frame 310 (I(0)) and continuing through image frames 310 (I(1) . . . , I(n−1), I(n)). The image frames 310 are acquired by a traversing imaging system 100, as discussed with respect to FIGS. 1, 2, and 7. Image frame 310 (I(n)) represents a current image, image frame 310 (I(n−1)(represents a previous image frame 310, and so on. The dimensions of the individual image frames 310 depicted in FIG. 3 are designated as Wo×Ho. Similarly, the dimension of the combination of the plurality of overlapping image frames 310 (e.g., an original panoramic image 320) depicted in FIG. 3 is designated with a size of (Wp×Wo). FIG. 3 also depicts a X-axis and Y-axis indicating a traversing direction (e.g., the X-axis) and non-traversing direction (e.g., the Y-axis) of the support gantry 102 during operation of the present invention.

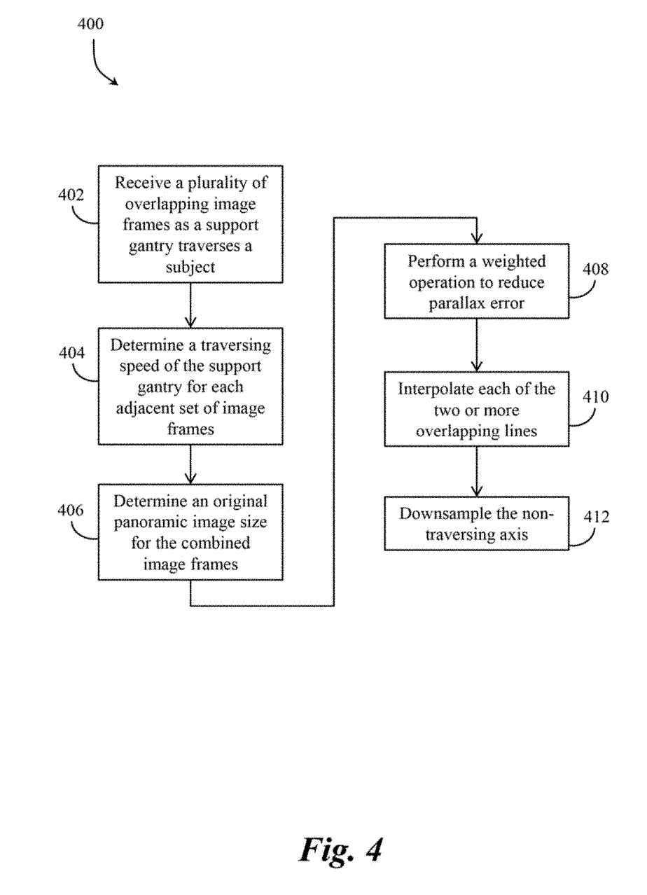

In operation, the stitching tool 216 utilizes a plurality of overlapping image frames 310, such as the image frames 310 depicted in FIG. 4 to create a single panoramic image 340 constructed in real-time from the overlapping image frames 310. To reduce parallax errors and display the panoramic image 340 in real-time, the stitching tool 216 performs a downsampling blending process. In accordance with an example implementation of the present invention, the stitching tool 216 utilizes the process 400 from FIG. 4 to perform the stitching process. In particular, FIG. 4 depicts a process implemented by the processing and display device 116 by executing the stitching tool 216 to stitch together a plurality of overlapping image frames 310. Initially, at step 402, the processing and display device 116 receives a plurality of overlapping image frames 310, such as the image frames 310 as depicted in FIG. 3, in real-time as the support gantry 102 traverses over a subject 114.

At step 404 the stitching tool 216 determines a traversing speed (e.g., via the tracking wheels 120) of the support gantry 102 for each adjacent set of image frames 310. In particular, the stitching tool 216 calculates a motion dx(n) along X-axis between current frame I(n) and previous frame I(n−1). As would be appreciated by one skilled in the art, this process is repeated for each subsequent image frame 310 and each preceding image frame 310 (e.g., dx(n), dx(n−1), dx(n−2), . . . etc.) using motion data obtained from the tracking wheels 120. In accordance with an example embodiment of the present invention, the motion dx(n) is determined based on the tracking information provided by the tracking wheels 120 (or one of the tracking wheels 120). As would be appreciated by one skilled in the art, because the support gantry 102 traverses a single axis (e.g., the X-axis), the motion change along Y-axis does not need to be calculated.

At step 406 the stitching tool 216 determines an original panoramic image size 320 for the combined plurality of image frames 310. In particular, the stitching tool 216 determines the original panoramic image size 320 by utilizing the motion data collected in step 404. In accordance with an example embodiment of the present invention, the motion data (e.g., dx(n), dx(n−1), dx(n−2), . . . , dx(1),) is utilized within an algorithm: Wₚ=Max{Σᵢⁿdx(i)}−Min{Σᵢⁿdx(i)}+Wₒ to determine the original panoramic image size 320. As would be appreciated by one skilled in the art, any combination of algorithms can be utilized in determining the dimensions of the original panoramic image size 320, without departing from the scope of the present invention. In the example of FIG. 3, the original panoramic image size 320 is Wp×Ho. As would be appreciated by one skilled in the art, because the support gantry 102 only traverses in a single axis, the dimension for the other axis is known (e.g., based on individual image frame dimensions). In FIG. 3, the constant axis is represented by dimension Ho.

At steps 408-410 the stitching tool 216 performs a downsampling process on the original panoramic image 320 along the traversing axis of the support gantry 102 (e.g., the X-axis direction) to obtain Pm(n) 330. At step 408 the stitching tool 216 performs a weighting operation to reduce parallax error. In particular, the stitching tool 216 selects the nearest lines (Li(0), Li(1), . . . , Li(N)) corresponding to the individual image frames I(x) (e.g., I(0) . . . I(n)) and assigns Li(k) to a Gaussian weight Wi(k) in the weighted algorithm of: Wᵢ₍ₖ₎=e^{-0.5(Dist(k)/64)}^{²}. Dist(k) identifies the distance between Li and Li(k) based on the traversing speed of the imaging system 100 (e.g., determined from the tracking wheels 120).

At step 410, based on the results of the weighting in step 408, the stitching tool 216 interpolates each line Li(k) to Lpi(k) to obtain the result line Li (as depicted in Pm(n) 330 of FIG. 3) after normalizing and summing all lines Lpi(k). As would be appreciated by one skilled in the art, any combination of interpolation and normalization methods can be utilized herein. As a result of the steps 408-410, the stitching tool 216 derives Pm(n) 330 including the line Li with the dimensions of Wd×Ho, as depicted in FIG. 3).

At step 412 the stitching tool 216 performs downsampling on Pm(n) 330 along the non-traversing axis (e.g., the Y-axis direction). Unlike in steps 408-410, there is no weight assignment needed when downsampling in the non-traversing axis because no motion occurred in that direction. In accordance with an example embodiment of the present invention, and to improve efficiency, the stitching tool 216 transposes the image frames 310 to calculate non-traversing axis motion and blend with the same method discussed with respect to steps 406-408. The result of the downsampling in the non-traversing axis is the final displaying panoramic image 340 (to be displayed to a user by the processing and display device 116). FIG. 3 depicts Disp(n) representing the final displaying panoramic image 340 resulting from the steps 402-412 (e.g., downsampling blending when stitching image frames 310). The resulting panoramic image 340 is much smaller than the original panoramic image 320 which, as depicted in FIG. 3, has dimensions of Wd×Hd.

In accordance with an example embodiment of the present invention, the processing and display device 116, as implemented by the correlation tool 218, applies a weighting blending profile (e.g., triangular or Gaussian weighting) to the stitching process. The weighted blending is the contribution factor of a pixel in a sub-image to panoramic/stitching image, as discussed in greater detail with respect to FIGS. 5 and 6.

FIG. 5 depicts two image frames 310 from a plurality of image frames 310 obtained as discussed with respect to FIGS. 1, 2, and 7. In particular, FIG. 5 depicts a first image frame 310a obtained by the processing and display device 116 at a first point in time and a second image frame 310b obtained by the processing and display device 116 at a second point in time. FIG. 5 also depicts a X-axis and Y-axis indicating a traversing direction (e.g., the X-axis) and non-traversing direction (e.g., the Y-axis) of the support gantry 102 during operation of the present invention. The image frame 310b (or I(n)) represents an image frame 310 at a current point in time while the image frame 310a (or I(n−1)) represents an image frame 310 at the previous point in time to the image frame 310b. The motion of the support gantry 102 between the image frame 310a and the image frame 310b is designated by the dx(n)′ value. Additionally, as reflected in FIG. 5, the image frames 310a and 310b have an overlapping area reflected by the shaded area.

FIG. 6 depicts the process 600 for identifying correlations between two overlapping image frames 310. At step 602 the correlation tool 218 identifies the overlapping areas for two adjacent image frames 310. In accordance with an example embodiment of the present invention, the overlapping areas are identified based on a determination of the motion of the support gantry 102 (dx(n)) between captures of the image frames and a known image frame size. As discussed herein, the motion dx(n) can be calculated based on the amount of time/traverse speed of the tracking wheels 120 or other method known in the art. In the example depicted in FIG. 5, the overlapping areas of the image frames 310a and 310b is represented by the shaded area of each respective image frame 310a, 310b.

At step 604 the correlation tool 218 selects a pattern image 510 within the overlapping area of the first image frames 310a (or I(n−1)) for correlation identification. The pattern image 510 is an area smaller than the overlapping area and is designed to improve the efficiency of the correlation search (rather than searching the entirety of the overlapping areas). As would be appreciated by one skilled in the art, the size of the pattern image 510 can be automatically determined by the correlation tool 218 or it can be a user defined value. In the example depicted in FIG. 5, the pattern image 510 is designed in image frame 310a as P(n−1).

At step 606 the correlation tool 218 searches the second image frame 310b (or I(n)/current image frame 310) for a pattern image 520 (or P′(n)) that mostly closely represents the pattern image 510 from the image frame 310a by comparing a correlation value. In accordance with an example embodiment of the present invention, the searching performed in step 606 is limited to a search area 530 which is an area within the overlapping area with a smaller dimension. As would be appreciated by one skilled in the art, the size of the search area 530 can be automatically determined by the correlation tool 218 or it can be a user defined value. For example, the search area 530 can be determined based on a current image frame rate and the tracking wheel 120 speed. By restricting the search to the search area 530, the correlation tool 218 is able to more efficiently identify the correlated areas. In an example, the image frame 310 size is 1024 pixel(width) by 1024 pixel(height), the pattern image 510 size is 256 pixel(width) and 256 pixel(height), and the search area 530 size is 512 pixel(width)×512 pixel(height).

In accordance with an example embodiment of the present invention, the correlation tool 218 utilizes the following algorithm to calculate the correlation between the pattern image 510 (P(n−1)) and the pattern image 520 (P′(n)) in the current image frame 310b. (I(n)):

$$Cor = \frac{{\sum }_{i = 0}^{N} {P}_{n - 1} ( i ) * {P}_{n}^{′} ( i )}{\sqrt{{\sum }_{i = 0}^{N} {P}_{n - 1} ( i ) * {P}_{n - 1} ( i )} \sqrt{{\sum }_{i = 0}^{N} {P}_{n}^{′} ( i ) * {P}_{n}^{′} ( i )}}$$

The resulting Cor value can range from 0 to 1.0, while the closer that the Cor value is to 1.0, the higher correlation exists between the two patterns/areas. In the algorithm, N is the pixel number in the pattern area, Pₓ(i) or Pₓ′(i) identifies the pixel value in the image frame 310 (I(x)). As would be appreciated by one skilled in the art, any combination of correlation algorithms can be utilized without departing from the scope of the present invention.

As depicted in FIG. 5, Cn(x, y) is the center of the Pn(x, y) area which is most likely to the pattern area 510 (P(n−1)). However, its accuracy is in pixel. Thus, to improve the accuracy, at step 608, the correlation tool 218 utilizes an interpolation method to get the final CFn(x, y) in sub pixel accuracy as follow:

$${CF}_{n} ( x , y ) = ( x + \frac{- 05. * ( {Cor}_{Right} - {Cor}_{Left} )}{( {Cor}_{Right +} {Cor}_{Left} - 2 * Cor )} , y )$$

$${Cor}_{Right} = \frac{{\sum }_{i = 0}^{N} {P}_{n - 1} ( i ) * {P}_{Right} ( i )}{\sqrt{{\sum }_{i = 0}^{N} {P}_{n - 1} ( i ) * {P}_{n - 1} ( i )} \sqrt{{\sum }_{i = 0}^{N} {P}_{Right} ( i ) * {P}_{Right} ( i )}}$$

$${Cor}_{Left} = \frac{{\sum }_{i = 0}^{N} {P}_{n - 1} ( i ) * {P}_{Left} ( i )}{\sqrt{{\sum }_{i = 0}^{N} {P}_{n - 1} ( i ) * {P}_{n - 1} ( i )} \sqrt{{\sum }_{i = 0}^{N} {P}_{Left} ( i ) * {P}_{Left} ( i )}}$$

Where P_{Right}(i) with center Cₙ(x+1,y), and P_{Left}(i) with center Cₙ(x−1,y). At step 610, after calculating the sub pixel CFn(x,y), the correlation tool 218 calculates motion dx(n) for sub pixel accuracy using the following algorithm: dx(n)=CFₙ(x)−CFₙ₋₁(x).

Utilizing the above-noted correlation identification, weighted blending, and stitching methodologies and system, the imaging system 100 is able to produce the single non-parallax panoramic image 340 in real-time (generated and updated as the support gantry 102 moves) for use during a fluoroscopic procedure. As such, the present invention provides an improvement in the functioning of the computer itself in that it enables the real-time stitching and displaying of images without requiring downsampling. The present invention also thereby is an improvement to existing digital medical image processing technologies. In accordance with an example embodiment of the present invention, the stitching method to produce the panoramic image is fully automated without any user input required. The stitching image frames 310 together and displaying the stitched panoramic image in real-time while the support gantry 102 traverses along the subject 114. In accordance with an example embodiment of the present invention, as raw image data/image frames 310 are received by the processing and display device 116, the panoramic image 340 is updated on the fly to produce the real time display. As would be appreciated by one skilled in the art, the stitching can be performed utilizing any stitching methods and systems known in the art to combine a plurality of images into a single image (e.g., through interpolating, blending, etc.).

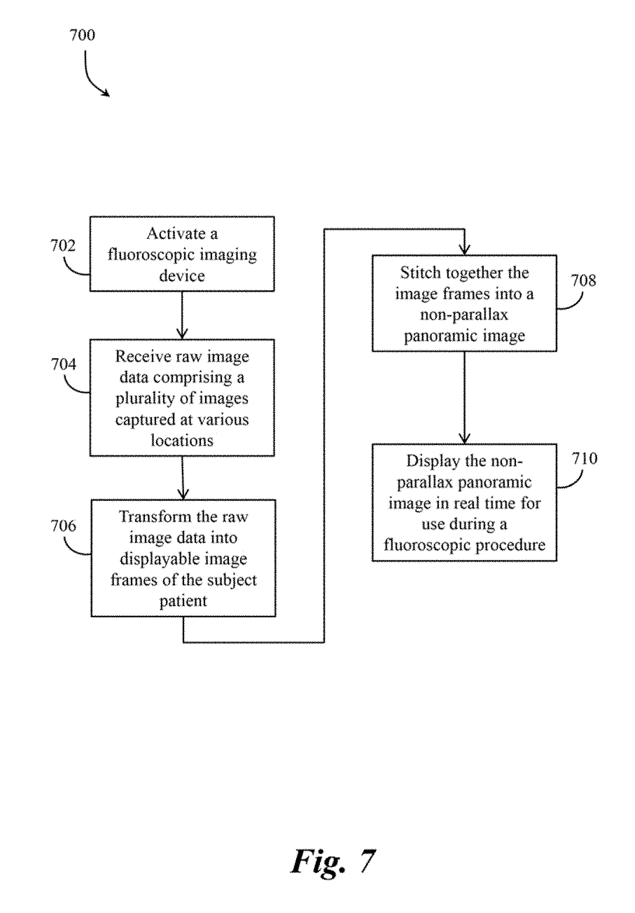

FIG. 7 depicts an example overall operation of the imaging system 100 in accordance with the present invention. In particular, FIG. 7 depicts a process 700 operation for utilization of a fluoroscopic imaging system. At step 702 a fluoroscopic imaging system 100, as discussed with respect to FIGS. 1-6, is activated. At step 704 a processing and display device 116 receives raw image data including a plurality of limited field of view image frames 310, each captured at the position of the support gantry 102 relative a subject 114 patient located between the radiation sources 104, 108 and the radiation detectors 106, 110. Additionally, the position of the support gantry 102 during the image capture is obtained by the processing and display device 116 (e.g., based on motion data from the tracking wheels 120). At step 706 the processing and display device transforms the raw image data into displayable image frames 310 of the subject patient on the fly. At step 708 the processing and display device 116 stitches together the displayable images, based on the position of the support gantry 102, into a non-parallax panoramic image 340. At step 710 the processing and display device displays the non-parallax panoramic image (e.g., panoramic image 340) to a user in real time (e.g., for use during a fluoroscopic procedure). Relying on the real-time panoramic image, the user can perform a fluoroscopic procedure, which reduces a radiation dosage applied to the patient.

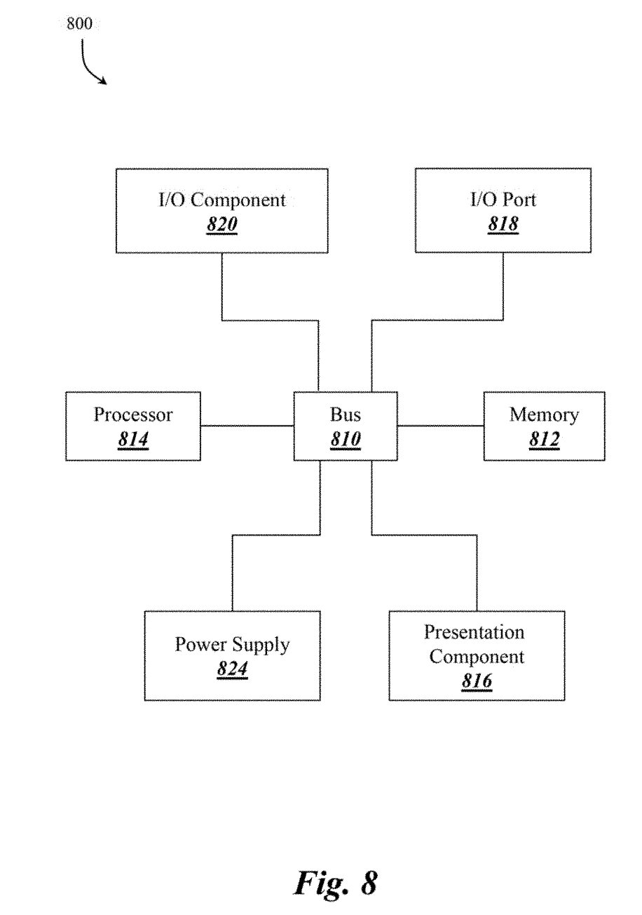

Any suitable computing device can be used to implement the computing devices (e.g., processing and display device 116) and methods/functionality described herein and be converted to a specific system for performing the operations and features described herein through modification of hardware, software, and firmware, in a manner significantly more than mere execution of software on a generic computing device, as would be appreciated by those of skill in the art. One illustrative example of such a computing device 800 is depicted in FIG. 8. The computing device 800 is merely an illustrative example of a suitable computing environment and in no way limits the scope of the present invention. A “computing device,” as represented by FIG. 8, can include a “workstation,” a “server,” a “laptop,” a “desktop,” a “hand-held device,” a “mobile device,” a “tablet computer,” or other computing devices, as would be understood by those of skill in the art. Given that the computing device 800 is depicted for illustrative purposes, embodiments of the present invention may utilize any number of computing devices 800 in any number of different ways to implement a single embodiment of the present invention. Accordingly, embodiments of the present invention are not limited to a single computing device 800, as would be appreciated by one with skill in the art, nor are they limited to a single type of implementation or configuration of the example computing device 800.

The computing device 800 can include a bus 810 that can be coupled to one or more of the following illustrative components, directly or indirectly: a memory 812, one or more processors 814, one or more presentation components 816, input/output ports 818, input/output components 820, and a power supply 824. One of skill in the art will appreciate that the bus 810 can include one or more busses, such as an address bus, a data bus, or any combination thereof. One of skill in the art additionally will appreciate that, depending on the intended applications and uses of a particular embodiment, multiple of these components can be implemented by a single device. Similarly, in some instances, a single component can be implemented by multiple devices. As such, FIG. 8 is merely illustrative of an exemplary computing device that can be used to implement one or more embodiments of the present invention, and in no way limits the invention.

The computing device 800 can include or interact with a variety of computer-readable media. For example, computer-readable media can include Random Access Memory (RAM); Read Only Memory (ROM); Electronically Erasable Programmable Read Only Memory (EEPROM); flash memory or other memory technologies; CDROM, digital versatile disks (DVD) or other optical or holographic media; magnetic cassettes, magnetic tape, magnetic disk storage or other magnetic storage devices that can be used to encode information and can be accessed by the computing device 800.

The memory 812 can include computer-storage media in the form of volatile and/or nonvolatile memory. The memory 812 may be removable, non-removable, or any combination thereof. Exemplary hardware devices are devices such as hard drives, solid-state memory, optical-disc drives, and the like. The computing device 800 can include one or more processors that read data from components such as the memory 812, the various I/O components 816, etc. Presentation component(s) 816 present data indications to a user or other device. Exemplary presentation components include a display device, speaker, printing component, vibrating component, etc.

The I/O ports 818 can enable the computing device 800 to be logically coupled to other devices, such as I/O components 820. Some of the I/O components 820 can be built into the computing device 800. Examples of such I/O components 820 include a microphone, joystick, recording device, game pad, satellite dish, scanner, printer, wireless device, networking device, and the like.

As utilized herein, the terms “comprises” and “comprising” are intended to be construed as being inclusive, not exclusive. As utilized herein, the terms “exemplary”, “example”, and “illustrative”, are intended to mean “serving as an example, instance, or illustration” and should not be construed as indicating, or not indicating, a preferred or advantageous configuration relative to other configurations. As utilized herein, the terms “about”, “generally”, and “approximately” are intended to cover variations that may existing in the upper and lower limits of the ranges of subjective or objective values, such as variations in properties, parameters, sizes, and dimensions. In one non-limiting example, the terms “about”, “generally”, and “approximately” mean at, or plus 10 percent or less, or minus 10 percent or less. In one non-limiting example, the terms “about”, “generally”, and “approximately” mean sufficiently close to be deemed by one of skill in the art in the relevant field to be included. As utilized herein, the term “substantially” refers to the complete or nearly complete extend or degree of an action, characteristic, property, state, structure, item, or result, as would be appreciated by one of skill in the art. For example, an object that is “substantially” circular would mean that the object is either completely a circle to mathematically determinable limits, or nearly a circle as would be recognized or understood by one of skill in the art. The exact allowable degree of deviation from absolute completeness may in some instances depend on the specific context. However, in general, the nearness of completion will be so as to have the same overall result as if absolute and total completion were achieved or obtained. The use of “substantially” is equally applicable when utilized in a negative connotation to refer to the complete or near complete lack of an action, characteristic, property, state, structure, item, or result, as would be appreciated by one of skill in the art.

Numerous modifications and alternative embodiments of the present invention will be apparent to those skilled in the art in view of the foregoing description. Accordingly, this description is to be construed as illustrative only and is for the purpose of teaching those skilled in the art the best mode for carrying out the present invention. Details of the structure may vary substantially without departing from the spirit of the present invention, and exclusive use of all modifications that come within the scope of the appended claims is reserved. Within this specification embodiments have been described in a way which enables a clear and concise specification to be written, but it is intended and will be appreciated that embodiments may be variously combined or separated without parting from the invention. It is intended that the present invention be limited only to the extent required by the appended claims and the applicable rules of law.

It is also to be understood that the following claims are to cover all generic and specific features of the invention described herein, and all statements of the scope of the invention which, as a matter of language, might be said to fall therebetween.

What is claimed is:

1. A method for panoramic imaging, the method comprising:
activating an imaging system, the imaging system comprising:
a first imaging assembly mounted on a support gantry, the first imaging assembly configured to capture image data comprising a plurality of image frames;
a control unit that directs movement and positioning of the support gantry; and
a processing and display device in communication with the first imaging assembly, the processing and display device configured to stitch and display the plurality of image frames as a single panoramic image; and
traversing the support gantry, via the control unit, parallel to a subject to be imaged;
wherein the processing and display device constructs and displays the single panoramic image in real-time based on the traversing of the support gantry and the plurality of image frames obtained during the traversing.

2. The method of claim 1, wherein the stitching comprises:
calculating a motion between image frames along an X-axis of the traversing the support gantry;
determining a size of an original panoramic image based on the calculated motion between image frames;
downsampling the original panoramic image along the X-axis;
determining a downsample size of the original panoramic image based on the downsampling; and
downsampling the original panoramic image along a Y-axis.

3. The method of claim 2, wherein the downsampling comprises:
selecting two or more overlapping lines between adjacent image frames;
determining a traversing speed of the support gantry;
performing a weighted operation to reduce parallax error;
interpolating each of the two or more overlapping lines; and
normalizing and summing the interpolated overlapping lines.

4. The method of claim 3, wherein the weighted operation is a Gaussian formula of:
Wᵢ₍ₖ₎=e^{-0.5(Dist(k)/64)}^{²}
where Wi(k) is a Gaussian weight and Dist(k) is a distance between overlapping lines for image frames.

5. The method of claim 2, wherein the downsampling is performed without a weighted operation.

6. The method of claim 1, wherein the stitching comprises identifying a correlation of overlapping region(s) from adjacent images to find an inter frame motion.

7. The method of claim 6, wherein identifying the correlation of overlapping region(s) comprises:
selecting a search area size for searching for a correlation within each of the plurality of image frames, wherein the search area size is smaller than a single image frame size;
selecting a pattern image size to be used in the correlation search, wherein the pattern image size is smaller than the search area size;
comparing a pattern image to an area defined by the search area size for each of the plurality of image frames; and
determining a correlation value based on the comparing.

8. The method of claim 1, wherein the first imaging assembly comprises a first imaging energy emitter that is positioned opposite a first imaging receptor, wherein one of the first imaging energy emitter or the first imaging receptor is positioned at a first terminal end of the support gantry.

9. The method of claim 8, wherein the imaging system further comprises a second imaging assembly that is positioned on the support gantry and comprising a second imaging energy emitter positioned that is opposite a second imaging receptor, wherein one of the second imaging energy emitter or the second imaging receptor is positioned at a second terminal end of the support gantry.

10. The method of claim 1, wherein the support gantry further comprises tracking wheels configured to enable traversing of the support gantry and tracking a distance traversed by the support gantry.

11. A system for panoramic imaging, the system comprising:
a support gantry including a plurality of tracking wheels configured to enable the support gantry to traverse in a single axis direction;
a first imaging assembly mounted on the support gantry, the first imaging assembly configured to capture image data comprising a plurality of image frames while the support gantry traverses parallel to a subject;
a control unit that directs movement and positioning of the support gantry; and
a processing and display device in communication with the first imaging assembly, the processing and display device configured to stitch and display the plurality of image frames as a single panoramic image; and
wherein the processing and display device is configured to construct and display the single panoramic image in real-time based on a traversing of the support gantry and the plurality of image frames obtained during the traversing.

12. The system of claim 11, wherein the stitching comprises:
the processing and display device calculating a motion between image frames along an X-axis of the traversing the support gantry;
the processing and display device determining a size of an original panoramic image based on the calculated motion between image frames;
the processing and display device downsampling the original panoramic image along the X-axis;
the processing and display device determining a downsample size of the original panoramic image based on the downsampling; and
the processing and display device downsampling the original panoramic image along a Y-axis.

13. The system of claim 12, wherein the downsampling comprises:
a stitching tool selecting two or more overlapping lines between adjacent image frames;
the stitching tool determining a traversing speed of the support gantry;
the stitching tool performing a weighted operation to reduce parallax error;
the stitching tool interpolating each of the two or more overlapping lines; and
the stitching tool normalizing and summing the interpolated overlapping lines.

14. The system of claim 13, wherein the weighted operation is a Gaussian formula of:
Wᵢ₍ₖ₎=e^{-0.5(Dist(k)/64)}^{²}
where Wi(k) is a Gaussian weight and Dist(k) is a distance between overlapping lines for image frames.

15. The system of claim 12, wherein the stitching tool performs the downsampling without a weighted operation.

16. The system of claim 11, wherein the stitching comprises a correlation tool identifying a correlation of overlapping region(s) from adjacent images to find an inter frame motion.

17. The system of claim 16, wherein identifying the correlation of overlapping region(s) comprises:
the correlation tool selecting a search area size for searching for a correlation within each of the plurality of image frames, wherein the search area size is smaller than a single image frame size;
the correlation tool selecting a pattern image size to be used in the correlation search, wherein the pattern image size is smaller than the search area size;
the correlation tool comparing a pattern image to an area defined by the search area size for each of the plurality of image frames; and
the correlation tool determining a correlation value based on the comparing.

18. The system of claim 11, wherein the first imaging assembly comprises a first imaging energy emitter that is positioned opposite a first imaging receptor, wherein one of the first imaging energy emitter or the first imaging receptor is positioned at a first terminal end of the support gantry.

19. The system of claim 18, wherein the system further comprises a second imaging assembly that is positioned on the support gantry and comprising a second imaging energy emitter positioned that is opposite a second imaging receptor, wherein one of the second imaging energy emitter or the second imaging receptor is positioned at a second terminal end of the support gantry.

20. The system of claim 11, wherein the plurality of tracking wheels are configured to track a distance traversed by the support gantry.
